(19) United States
(12) Patent Application Publication
(10) Pub. No.: US 2019/0225949 A1
(43) Pub. Date: Jul. 25, 2019

(54) Methods and Apparatus for Cell-Free Microfluidic-Assisted Biosynthesis

(71) Applicants: Massachusetts Institute of Technology, Cambridge, MA (US); Regents of the University of Minnesota, St. Paul, MN (US)

(72) Inventors: Andreas Mershin, Cambridge, MA (US); Vincent Noireaux, Minneapolis, MN (US); James Francis Pelletier, Cambridge, MA (US); Neil A. Gershenfeld, Cambridge, MA (US)

(73) Assignees: Massachusetts Institute of Technology, Cambridge, MA (US); Regents of the University of Minnesota, St. Paul, MN (US)

(21) Appl. No.: 16/137,536

(22) Filed: Sep. 20, 2018

Related U.S. Application Data
(62) Division of application No. 14/735,132, filed on Jun. 9, 2015, now abandoned.
(60) Provisional application No. 62/009,703, filed on Jun. 9, 2014.

(51) Int. Cl. C12N 9/10; C12P 21/02; C12M 1/00

(52) U.S. Cl. CPC C12N 9/1029; C12P 21/02; C12M 47/06; C12Y 203/01; C12M 47/10

(57) ABSTRACT

A trans-disciplinary system for cell-free biosynthesis includes a cell-free transcription-translation (TX-TL) tool and modular, generalizable microfluidic architectures. Both components of the system are independently functional and are combinable into a cell-free biosynthesis platform. In the first component, modular plasmid libraries are used to program bacterial cell-free TX-TL systems. Each plasmid holds one gene or operon, and all the genes are controlled by the same promoter, so that the stoichiometry of enzyme synthesis is determined by the stoichiometry of plasmids in the reaction. In the second part, in order to facilitate high throughput mixing and matching of gene units from the modular plasmid libraries, a modular, reconfigurable, flexible, and scalable microfluidic architecture is employed. The microfluidic modules share common form factors and port/valve locations, so that a small set of module types, with multiple instances of each type interconnected in different geometries, allows simple reconfiguration to achieve different modes of operation.

### RELATED APPLICATIONS

This application is a divisional of U.S. patent application Ser. No. 14/735,132, filed Jun. 9, 2015, which claims the benefit of U.S. Provisional Application Ser. No. 62/009,703, filed Jun. 9, 2014, the entire disclosures of which are herein incorporated by reference.

### FIELD OF THE TECHNOLOGY

The present invention relates to molecular biology apparatus and protocols and, in particular, to synthesis of biosynthetic products.

### BACKGROUND

Due to the complexity of biosynthetic pathways, current strategies for synthesis of biosynthetic products fail to enable control over the diversity and yield of such products.

### SUMMARY

In one aspect, the invention enables rapid design-build-test cycles of metabolic pathways, in order to optimize synthesis of known biosynthetic products (such as, but not limited to, anticancer drugs and antibiotics) and to facilitate systematic searches through gene sets for new biosynthetic products.

In particular, the invention comprises a trans-disciplinary strategy combining two novel approaches: 1) a cell-free transcription-translation (TX-TL) tool harnessing the power of a novel gene expression library, and 2) uniquely modular and generalizable microfluidic architectures. Both components of the system work independently of one another, and they are leveraged by one another when deployed in combination. While independently functional, the two components can also be combined into a platform capable of testing hundreds to thousands of biosynthesis conditions per day by continuous perturbation/permutation of physicochemical parameters (combinatorial biochemistry).

In a preferred embodiment, the invention employs all-E. coli transcription-translation (TX-TL) in cell-free bacterial extract with modular plasmid libraries and microfluidic devices. The invention optimizes biosynthesis of desired products in cell-free TX-TL systems.

In some exemplary implementations of this invention, a cell-free transcription-translation (TX-TL) tool constructs a gene expression library that allows control over relative promoter strength without using different promoters.

In some exemplary implementations of this invention, hardware components comprise a modular, reconfigurable, flexible, scalable and generalizable microfluidic architecture. The microfluidic feature's and access ports and control valving, are such that modules share a common form factor, including port and valving connector locations. A small set of module types are used, with multiple instances of each type interconnected in different geometries (for instance, but not limited to, parallel, series, or cascading). This permits simple reconfiguration in order to achieve different modes of operation.

In some exemplary implementations, this invention may be used to produce known biosynthetic products (such as cancer drugs) or to conduct systematic searches for new biosynthesis products having pre-determined properties.

In some exemplary implementations, this invention performs cell-free synthesis using modular microfluidics. This facilitates biosynthetic mass production and fast searches for new pathways expressing products with user specified properties.

### BRIEF DESCRIPTION OF THE DRAWINGS

Other aspects, advantages and novel features of the invention will become more apparent from the following detailed description of the invention when considered in conjunction with the accompanying drawings wherein:

FIG. 1 illustrates gene set library design using an exemplary cell-free TX-TL circuit, according to one aspect of the invention;

FIG. 2 illustrates transcript abundance via promoter strength according to an exemplary implementation of one aspect of the invention;

FIG. 3 illustrates transcript abundance via plasmid copy number, according to an alternative exemplary implementation of one aspect of the invention;

FIG. 4 depicts an exemplary microfluidic architecture, according to an aspect of the invention;

FIG. 5 depicts an exemplary implementation of a microfluidic TX-TL chamber, according to an aspect of the invention;

FIG. 6 depicts an exemplary two-component system according to an aspect of the invention;

FIG. 7 depicts an exemplary implementation of a microfluidic TX-TL system, according to an aspect of the invention;

FIG. 8 illustrates cell-free transcription-translation (TX-TL) droplet composition in an exemplary implementation of the invention;

FIG. 9 presents an example of integrated modular microfluidics with automated assay, with programmed droplet generation and manipulation modules, connected on chip, according to an exemplary embodiment;

FIG. 10 is a block diagram depicting hardware interconnectivity for an exemplary implementation of the invention

FIG. 11 depicts an exemplary implementation of one module of 20 TX-TL chambers, according to one aspect of the invention;

FIG. 12 depicts an exemplary implementation of a cell-free TX-TL circuit, suitable for iterative optimization for a two step biosynthesis pathway for green fluorescent protein;

FIG. 13 is a 3D plot of deGFP final yield tuned as a function of two concentrations, using the TX-TL circuit of FIG. 12;

FIG. 14 is an operational flowchart of an exemplary embodiment of a two-component system, according to one aspect of the invention; and

FIGS. 15A-B illustrate the independence of the two components of the system, wherein FIG. 15A depicts the options for an exemplary implementation of a cell-free TX-TL system according to one component of the system and FIG. 15B depicts options for an exemplary implementation of microfluidic cell-free biosynthesis according to the other component.

### DETAILED DESCRIPTION

The present invention leverages emergent-knowledge in cell-free synthesis and microfluidics to solve the problem of lack of control over diversity and yield during synthesis of biosynthetic products. In a particular aspect, the invention comprises a trans-disciplinary strategy combining two novel approaches: 1) a cell-free transcription-translation (TX-TL) tool harnessing the power of a novel gene expression library, and 2) uniquely modular and generalizable microfluidic architectures.

In a preferred embodiment of the invention, a library of polyketide synthase (PKS) gene units is built using a single gene promoter and expressed in a novel TX-TL system. This entirely new strategy allows fine-tuning of expression levels and leads to 100 times more efficient iterative yield optimization. A modular microfluidic platform developed using the unique capability to direct-write via laser micromachining optimizes biosynthesis by heterologous expression in vivo or by traditional cell-free expression, so both aspects of the invention work independently of each other and are leveraged by each other when deployed in combination. Specifically, while independently functional, the two new technologies can also be combined into a platform capable of testing hundreds to thousands of biosynthesis conditions per day by continuous perturbation/permutation of physicochemical parameters. Throughout, the invention follows digital material design principles emphasizing component modularity and system adaptability at low cost.

In the first part of the two components of the system of the invention, modular plasmid libraries are used to program bacterial cell-free TX-TL systems. Each plasmid holds just one gene or operon, and all the genes are controlled by the same promoter, so that the stoichiometry of enzyme synthesis is determined by the stoichiometry of plasmids in the reaction. In the second part, in order to facilitate high throughput mixing and matching of gene units from the modular plasmid libraries, a modular, reconfigurable, flexible, and scalable microfluidic architecture is employed. For example, microfluidic modules share common form factors and port/valve locations, so that a small set of module types, with multiple instances of each type interconnected in different geometries (e.g., parallel, series, or cascading), allows simple reconfiguration to achieve different modes of operation.

Construction of Gene Expression Library. In an example of this invention, a library of polyketide synthase (PKS) gene units is built using a single gene promoter and expressed in a TX-TL system. Expression levels are fine-tuned by manipulating the relative volumes of the PKS units that are deposited in the TX-TL reaction chamber. A single promoter, ribosome binding site, and transcriptional terminator, common to all gene units, may be used. Either plasmids or PCR (polymerase chain reaction) products may be employed. In this example, the library may be used for iterative optimization of polyketide production.

A set of plasmids specifically developed for the all E. coli TX-TL system is used. Genes are cloned either under E. coli regulatory parts (regulated or unregulated E. coli promoters) or under bacteriophage promoters (T7, T3 or SP6). Plasmids have antibiotic resistance (ampicillin, chloramphenicol) and origin of replication (ColE1, p15A) for amplification through E. coli.

In illustrative implementations of this invention, a library of PKS genes are all controlled by a single promoter. A collection of plasmids is generated. To vary biosynthesis conditions, the ratio of plasmids is adjusted. As a result, iterations can circumvent the laborious and costly cloning step. Ambiguity resulting from differences in the relative expressions of different promoters under different conditions may be eliminated. To enable massively-parallel testing of unlimited PKS levels, one can construct a library of PKS gene units (2-3 genes), all with identical regulatory parts. Then, in the cell-free TX-TL reaction, the concentration of the gene units will determine the concentration of the PKSs. Thus, PKS stoichiometry can be varied easily it becomes possible to explore many conditions in parallel and circumvent continued genetic manipulations. Then, in each droplet (if in a microfluidic setting) or in each Eppendorf tube if in bulk TX-TL setting, the PKS abundance is controlled by the easily measurable volume corresponding to gene copy number. For example, a spherical droplet (with diameter 10 microns and maximum concentration of each gene on the order of 10 nM) can include on the order of 100 different genes per droplet and between 0 and 50 copies of each gene. One example use is the MazF-MazE toxin-antitoxin pair from E. coli to modulate the global mRNA degradation rate; to first order. For that use, the amount of PKS is preferably proportional to the gene unit copy number.

Cell-free extract preparation. The cell-free TX-TL system is composed of an E. coli cytoplasmic extract containing the endogenous molecular machineries for coupled TX and TL. The cell-free TX-TL reaction is fueled by adding an energy mixture containing the four ribonucleosides and an ATP regeneration system. A mixture of the twenty amino acids is added for translation. The cell-free reaction is incubated between 29-37 C.

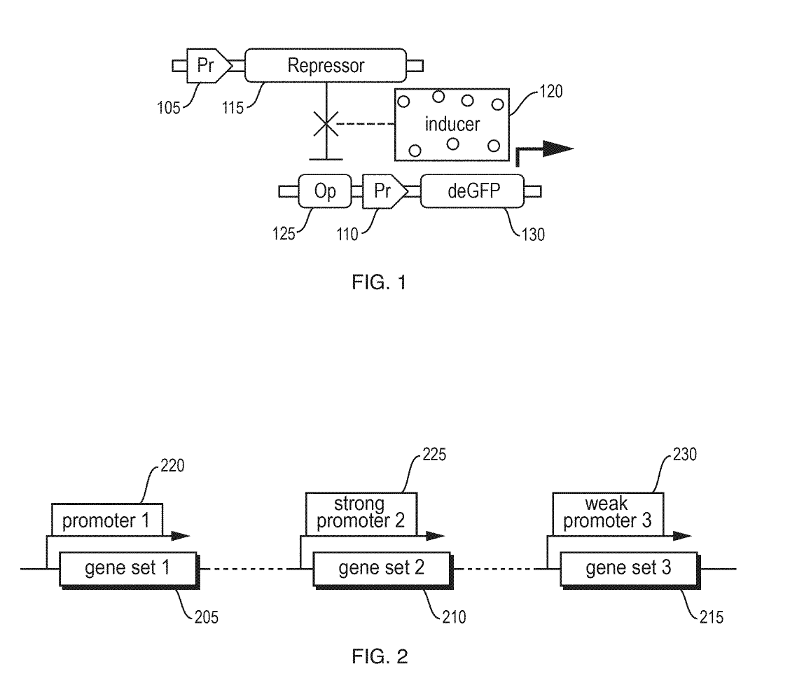

FIG. 1 illustrates gene set library design using cell-free TX-TL circuits, according to one aspect of the invention. Shown in FIG. 1 is a biosynthesis pathway for green fluorescent protein (GFP), including promotors 105, 110, repressor 115, and inducer 120, which together operate 125 to transcribe deGFP 130.

FIG. 2 illustrates transcript abundance via promoter strength according to an exemplary implementation of the invention. Shown in FIG. 2 are gene sets 205, 210, 215 and their respective promoters—average 220, strong 225, and weak 230.

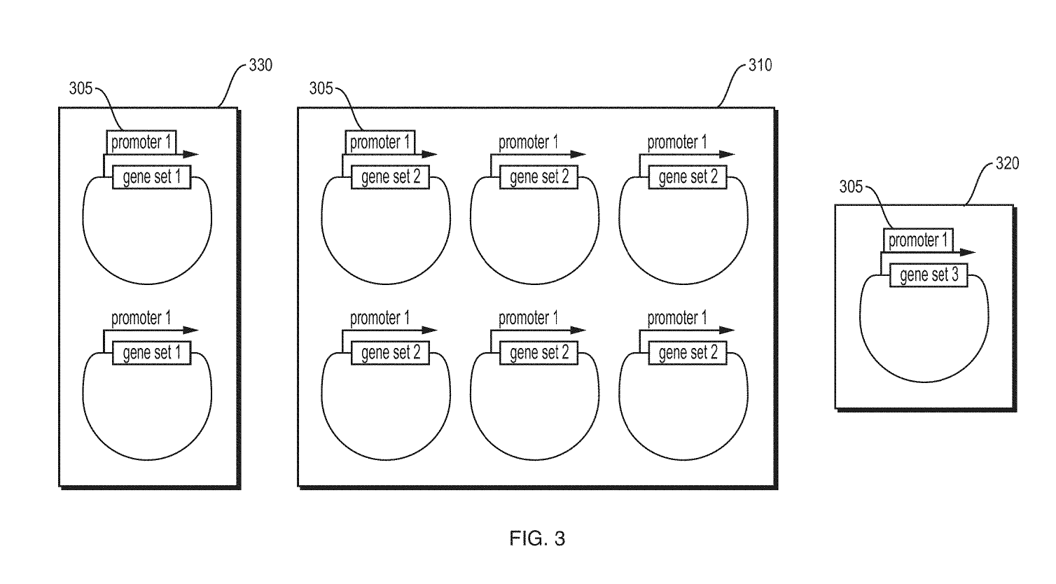

FIG. 3 illustrates transcript abundance via plasmid copy number, according to an alternative exemplary implementation of the invention. In FIG. 3, all plasmids have same promoter (promoter 1 305). Shown are most abundant plasmid 310, least abundant plasmid 320, and average abundance plasmid 330.

In general, the two-step procedure for this aspect of the invention is (1) build a set of plasmids, which takes approximately 2-3 days, with approximately one plasmid per gene, and identical regulatory parts (promoter, UTR, terminator). Next is (2) cell-free expression, which takes approximately 1-2 days, with unlimited stoichiometry and unlimited combinations. No cycling is required, as multi-parallel testing (in multi-well plates or microfluidics) can be utilized.

Modular Hardware Platform. In illustrative implementations of this invention, a modular droplet microfluidic platform is made using direct-write via excimer laser micromachining. Channels and features are be written on glass or silicon and sealed with flat PDMS (polydimethylsiloxane) or written directly on PDMS and sealed with flat glass or Silicon. For mass production master negative casts on glass or silicon can be cloned via PDMS into epoxy and used to make PDMS devices sealed by flat glass or silicon. Alternatively, traditional lithographic methods can be used to make the features on glass or Silicon and devices are then sealed with flat PDMS.

Microfluidic device design. To design microfluidic devices fabricated via soft lithography, CAD software is used to design chrome photomasks, and chrome masks are purchased from commercial vendors. The devices include features with various geometries and length scales, from microns to centimeters. Positive (e.g., phenolic resins)/negative (e.g., SU-8) photoresists are spin coated onto silicon wafers, and a mask aligner projects the image of the chrome mask onto the substrate to cure shielded/exposed regions of the photoresist layer, then the resist is developed with solvents. In some cases, the resist is baked before or after exposure. Soft lithography includes a wide range of subtractive and additive processes, combined to make devices with multiple layers; in one instance, Bosch deep reactive-ion etching is used (subtractive) to pattern shallow features (submicron to microns), such as chambers for cells and whole genomes; then SU-8 (additive) is used to pattern deeper features (microns to tens of microns), such as channels that connect the chambers to inlets, outlets, and various fluidic modules.

To design microfluidic devices fabricated via excimer laser micromachining, CAD/CAM software is used to generate toolpaths for the automated excimer stage. The excimer emits an energetic, pulsed UV beam (KrF 248 nm, ArF 193 nm) with a uniform profile, several centimeters in diameter. Masks are machined in stainless steel sheets, each on the order of 100 microns thick, and the masked beam is focused onto substrates such as borosilicate glass, silicon, polydimethylsiloxane (PDMS), polycarbonate, polyimide, and epoxies. The excimer ablates on the order of 100 nm depth per pulse and can achieve feature footprints with various shapes and sizes (microns to millimeters). In contrast to conventional soft lithography techniques, the excimer enables rapid design/build/test iteration of 2.5D devices in hard substrates.

Fabrication of microfluidic masters. To assemble the microfluidic devices, in one case, polydimethylsiloxane (PDMS) flow and control layers are cast from the masters fabricated via soft lithography. To prevent adhesion between the cured PDMS and the silicon or glass master, the master is vapor coated in an evacuated vacuum chamber with (tridecafluoro-1,1,2,2-tetrahydrooctyl)trichlorosilane (TFOCS) for about 2 hours at room temperature, to silanize the surfaces. The elastomer base and the curing agent are mixed in a 10:1 ratio by mass, the air bubbles removed with a vacuum pump or a centrifuge, and the viscous PDMS poured over the master. The PDMS is cured on the master at 65 to 80 C for about 12 hours, then the device is peeled from the master and left at room temperature. In another case, borosilicate glass cover slips with microfluidic features are used, such as those fabricated with the excimer laser, rather than PDMS slabs with microfluidic features. If the PDMS layer is not thick enough to support stable interfaces with tubing, PDMS blocks above inlet/outlet features are plasma bonded (protocol described below). Then a coring tool is used to punch inlet/outlet holes in the PDMS.

Next, the device components are bonded together. For example, if the device includes PDMS, oxygen plasma is used to bond textured PDMS to flat borosilicate cover slips, or textured cover slips to flat PDMS, or PDMS layers to one another; for example, the control layer to the flow layer. Several plasma systems can be used, each with its own optimal settings and occasional calibration required. For example, in one case, the substrates are placed onto a metal grid within a vacuum chamber, the chamber evacuated with a rotary vacuum pump for 1 minute, the vacuum chamber is put into a large microwave, and the microwave run at 30 percent power for 4 seconds. With these settings, the plasma sparks after about 2 seconds and glows purple. Then the surfaces are adhered without introducing air bubbles, and baked at 80 C for 10 minutes. In another case, borosilicate cover slips are cleaned and bonded together in a muffle furnace at 640 C for several hours. To interface with the device, NanoPorts™ (Upchurch) are used.

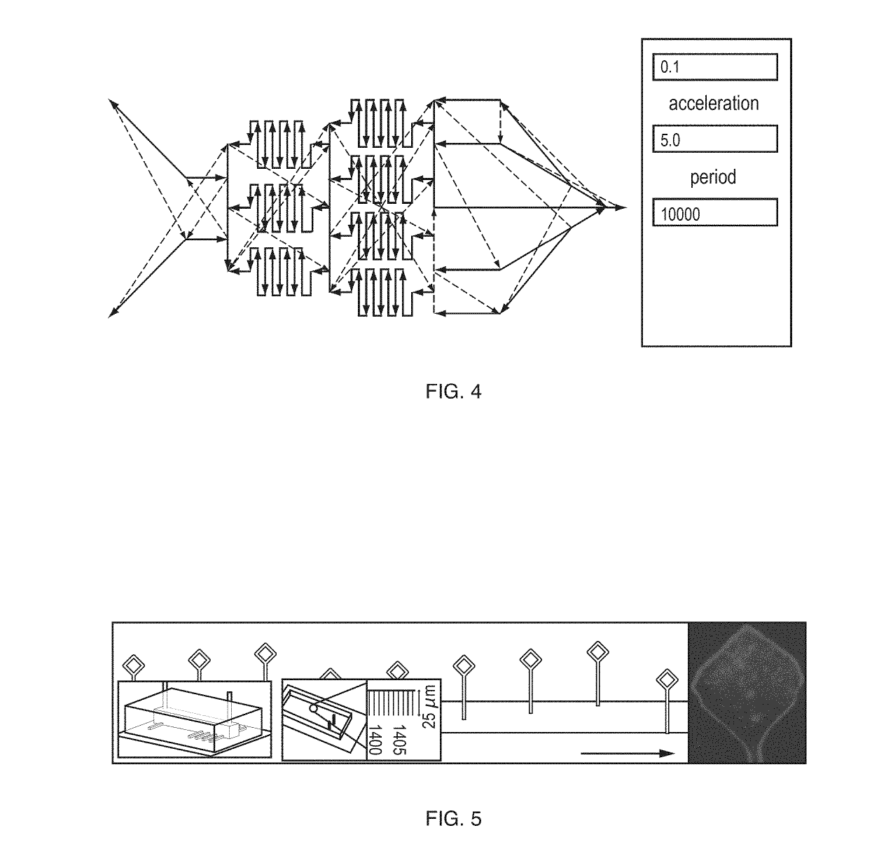

FIG. 4 depicts an exemplary microfluidic architecture, while FIG. 5 depicts an exemplary implementation of a microfluidic TX-TL chamber, according to an aspect of the invention.

Microfluidic device operation. To infuse buffers to the microfluidic devices, a syringe mounted on a syringe pump is used, and syringes are connected to the microfluidic device via polyethylene, Tygon, or some other tubing. Blunt needles or Upchurch fittings are used to connect the tubing to Luer-Lok or slip tip syringes.

Droplets of controlled volumes are generated from channels with each of the gene units, then droplets are fused to form a reaction volume with the desired abundance of each enzyme in the pathway. Progress of reactions are assayed in real time by an automated camera and microscope system.

Droplets are cell-free transcription-translation reactions surrounded by a carrier oil. For example, a fluorous carrier oil such as HFE7500 (Novec, 3M), with a surfactant to stabilize droplets such as Pico-Surf™ (Dolomite Microfluidics) is used. Droplets are dispensed in controlled amounts using microfluidic valves, in a control PDMS layer above and/or in the same layer as the extract-in-oil channels. Droplets with plasmids expressing different gene modules are combined in programmed stoichiometries. Then, they are fused via electrocoalescence or mechanical pressure and incubated in a specific region of the device.

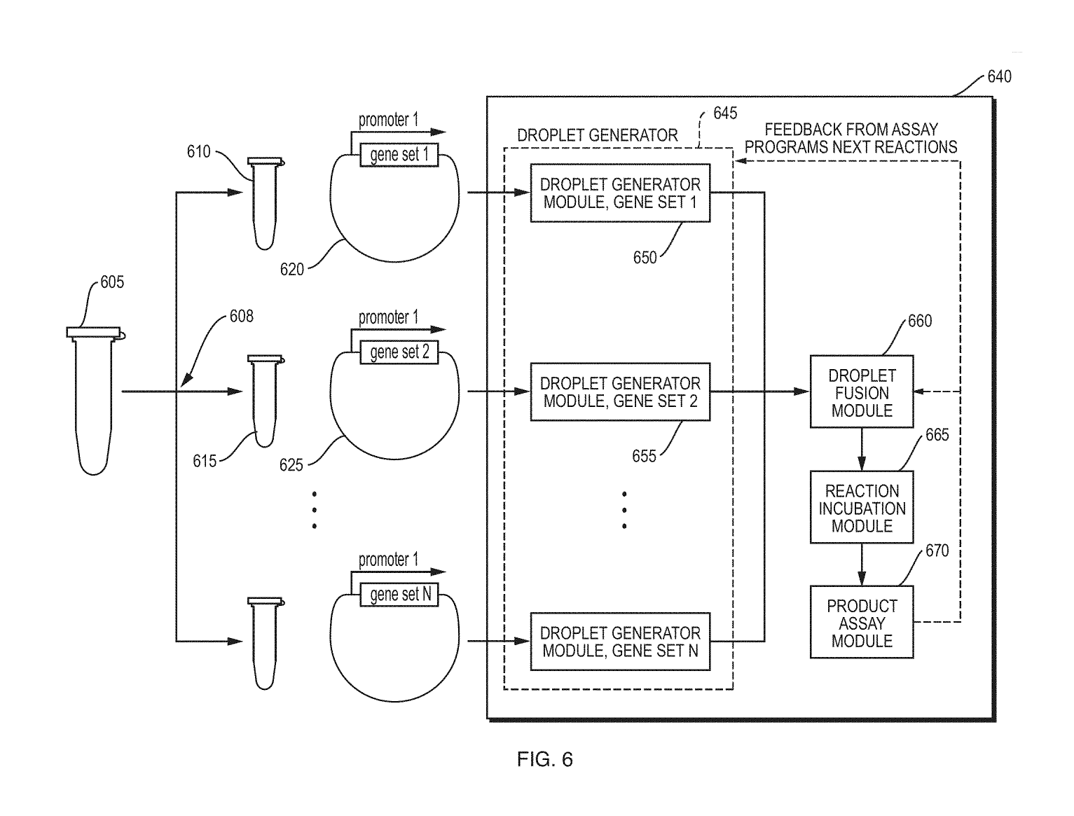

FIG. 6 depicts an exemplary two-component system according to an aspect of the invention. As seen in FIG. 6, cell-free extract 605 is split 608 into parts 610, 615 and plasmids 620, 625 are added. The individual extracts with plasmids enter the fluidic device 640, first into droplet generator 645 which has individual modules 650, 655 for each sample, then to droplet fusion module 660 reaction incubation module 665, and product assay module 670.

Individual devices may be interconnected with tubing and valves actuated by solenoid switches controlled by computer. Failure of one device does not cascade.

Droplets of equal volume are generated from containers with each of the PKS gene units and different number of droplets are added to achieve the desired levels of relative expression in each pathway. Each pathway's progress can be assayed in real time by an automated camera and microscope system and pathways in parallel, but running with lags in phase may also be employed in order to optimize each run.

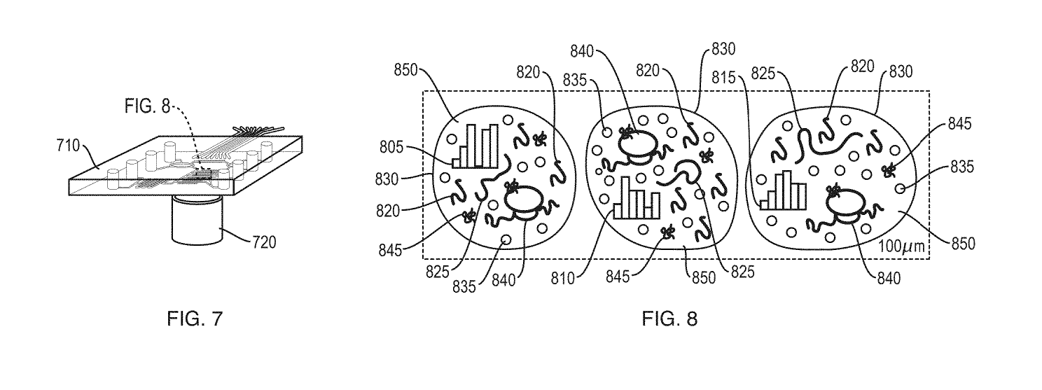

FIG. 7 depicts an exemplary implementation of a microfluidic TX-TL system, according to an aspect of the invention. Shown in FIG. 7 are integrated sensors 710 (high volume, low resolution) and automated microscope 720 (high resolution, low volume).

FIG. 8 illustrates cell-free transcription-translation (TX-TL) droplet composition in an exemplary implementation of the invention. Shown in FIG. 7 are bar graphs 805, 810, 815 of gene abundance, target molecules 820, mRNA 825, aqueous TX-TL droplets 830, small molecules 835, ribosomes 840, enzymes 845, and fluorinated carrier oil 850.

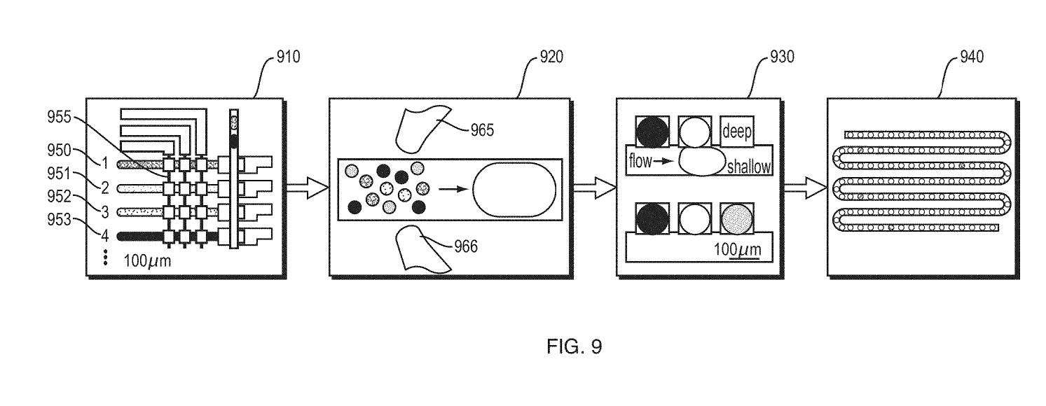

FIG. 9 presents an example of integrated modular microfluidics with automated assay, with programmed droplet generation and manipulation modules, connected on chip, according to an exemplary embodiment. Steps depicted in FIG. 9 are droplet generation 910, droplet fusion 920, electrodes, droplet storage 930, and performance assay 940. Also shown are representations of genes 950, 951, 952, 953, valves 955, and electrodes 965, 966.

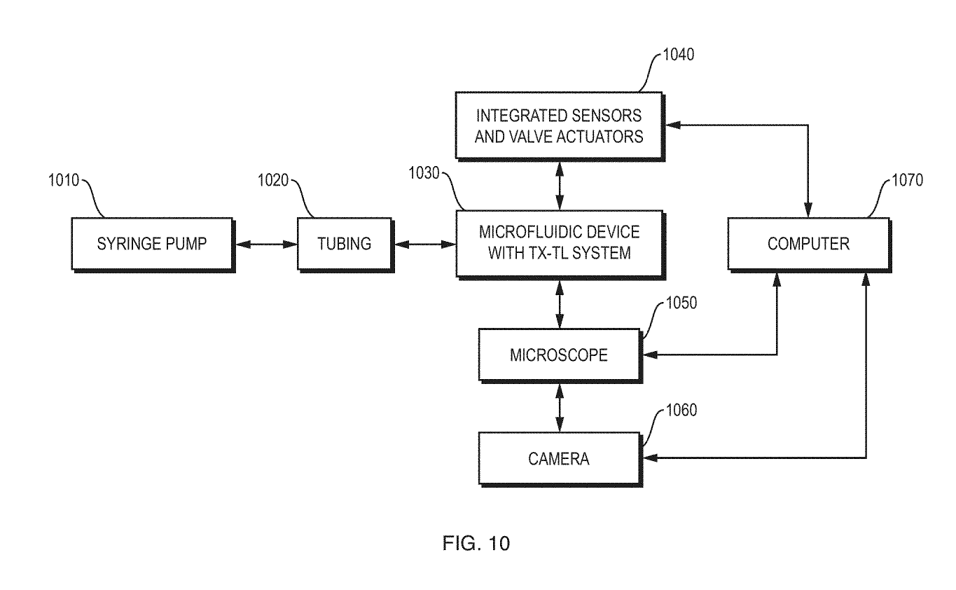

FIG. 10 is a block diagram depicting hardware interconnectivity for an exemplary implementation of the invention. Shown in FIG. 10 are syringe pump 1010, tubing 1020, microfluidic device with TX-TL system 1030, integrated sensors and valve actuators 1040, microscope 1050, camera 1060, and computer 1070. Fluorescence assays results from microscope 1050 are used as feedback to computer 1070.

FIG. 11 depicts an exemplary implementation of one module 1105 of 20 TX-TL chambers, according to one aspect of the invention. In the system of FIG. 11, valving and droplet generation are controlled by computer 1110 and assay results are fed back for continuous control.

Exemplary Operation Modes. This invention may be implemented in many different ways. Illustrative examples include, but are not limited to end-point yield optimization, real-time yield optimization, and mass production. The operation modes described herein are not the only way that this invention may be implemented.

End-point yield optimization, also called iterative yield optimization. In this mode, the invention optimizes yield of a known product, by adjusting the abundance of enzymes in the metabolic pathway to maximize desired fluxes through the pathway. The microfluidic modules are arranged such that yield of fully executed pathways is tracked as a function of the relative expression levels of each enzyme in the biosynthetic sequence.

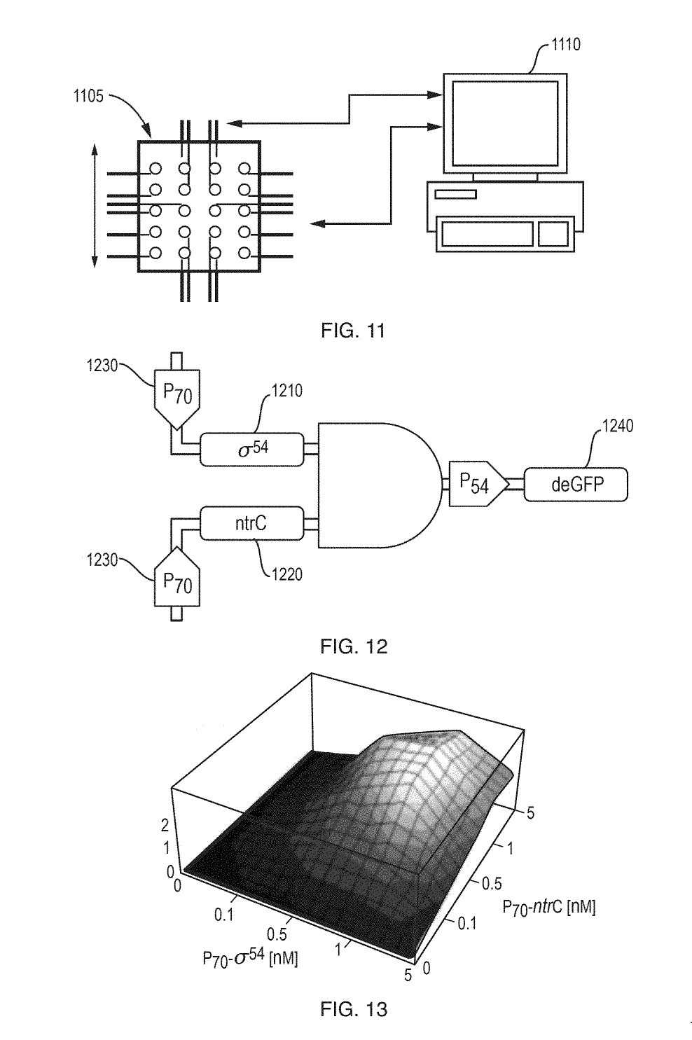

An example of this mode is shown in FIG. 12. FIG. 12 depicts an exemplary implementation of a cell-free TX-TL circuit, suitable for iterative optimization for a two-step biosynthesis pathway for green fluorescent protein (GFP). In this example, genes σ54 1210 and ntrC 1220 are cloned under promoter P70 1230 in separate plasmids and expressed concurrently to transcribe deGFP 1240. FIG. 13 is a 3D plot of deGFP final yield, tuned as a function of two concentrations, using the TX-TL circuit of FIG. 12.

For this example, N identical biosynthesis chambers may be loaded from two containers (one with filled with the product of the σ54 TX-TL reaction and the other with the product from the ntrC TX-TL reaction). N is the product of the number of desired concentration steps on each axis. The stoichiometry of σ54 to ntrC in each biosynthesis chamber can thus be varied and the resolution in both axes can be increased by simply tiling additional identical modules.

All modules are fed from the same two stock solutions but the number of droplets of each is varied. As the size of the droplets can also be controlled, coarse resolution can be achieved as easily as fine resolution, while keeping the total volumes within desired limits.

Real-time yield optimization. Real-time yield optimization is an automated real-time assay with, or without, feedback control. In this mode, reactions are monitored in real time, and the results of reactions are analyzed to inform the composition of subsequent reactions within the experiment. This mode may be used for monitoring both TX-TL reactions as they generate the enzymes for each step and also for monitoring the biosynthesis progress as products of each successive enzymatic reaction become substrates for the next. This is similar to end-point yield optimization; however, in this operation mode, the products are assayed as reactions progress and adjustments are made by the computer controlling the valving in real time. Reactions can be run in staggered mode so that the lessons learned from an earlier batch can be applied to optimize a later batch. This allows piece-meal optimization over multi step biosynthetic pathways.

Mass production. In this mode, once a set of condition has been found that is optimal, the same conditions are set as starting points for each reaction run and results are pooled

Separate or Combined Approaches. In exemplary implementations of this invention, the gene expression library and modular hardware architecture are combined into a platform capable of testing hundreds to thousands of biosynthesis conditions per day by continuous perturbation/permutation of physicochemical parameters.

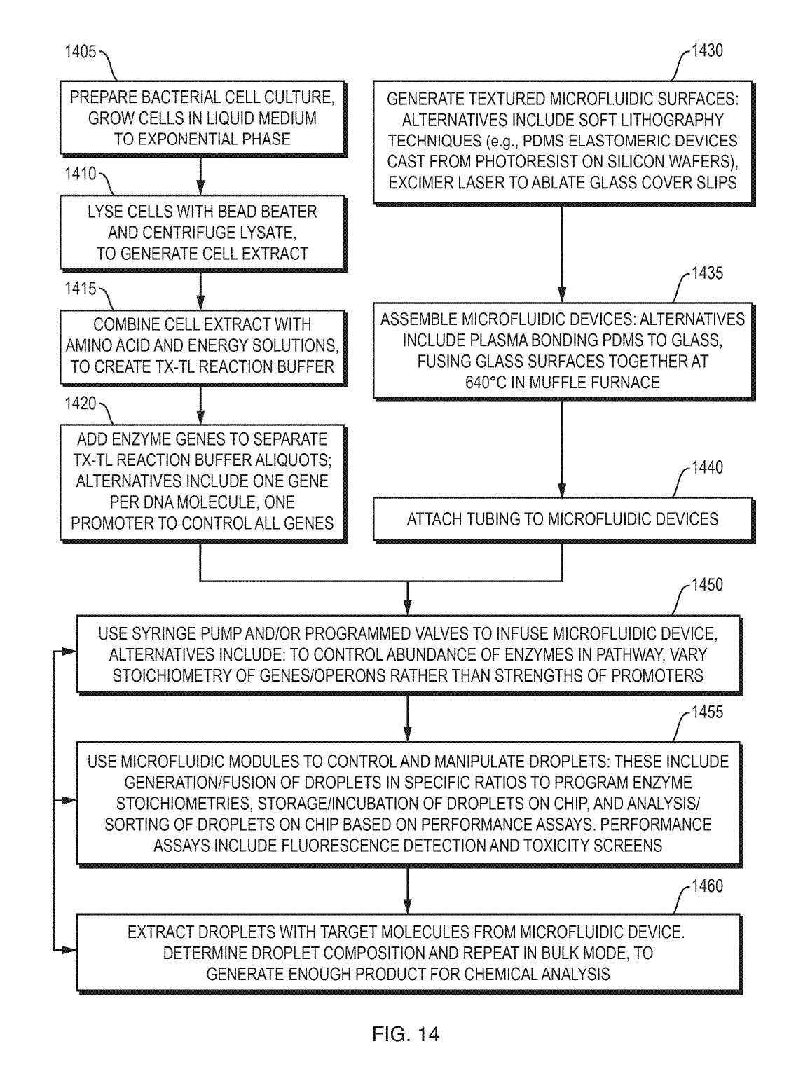

FIG. 14 is an operational flowchart of an exemplary embodiment of a two-component system, according to one aspect of the invention. In FIG. 14, the first component is implemented by preparing 1405 and growing the bacterial cell culture, generating 1410 the cell extract, creating 1415 the TX-TL reaction buffer, and adding 1420 the genes and promoters. The second component is implemented by generating 1430 the microfluidic surfaces, assembling 1435 the microfluidic devices, and attaching 1440 the tubing to the microfluidic devices. The two components are then combined to infuse 1450 the microfluidic device with the genes, generating, controlling, and manipulating 1455 droplets using the microfluidic modules, and extracting 1460 droplets with target molecules from the microfluidic device.

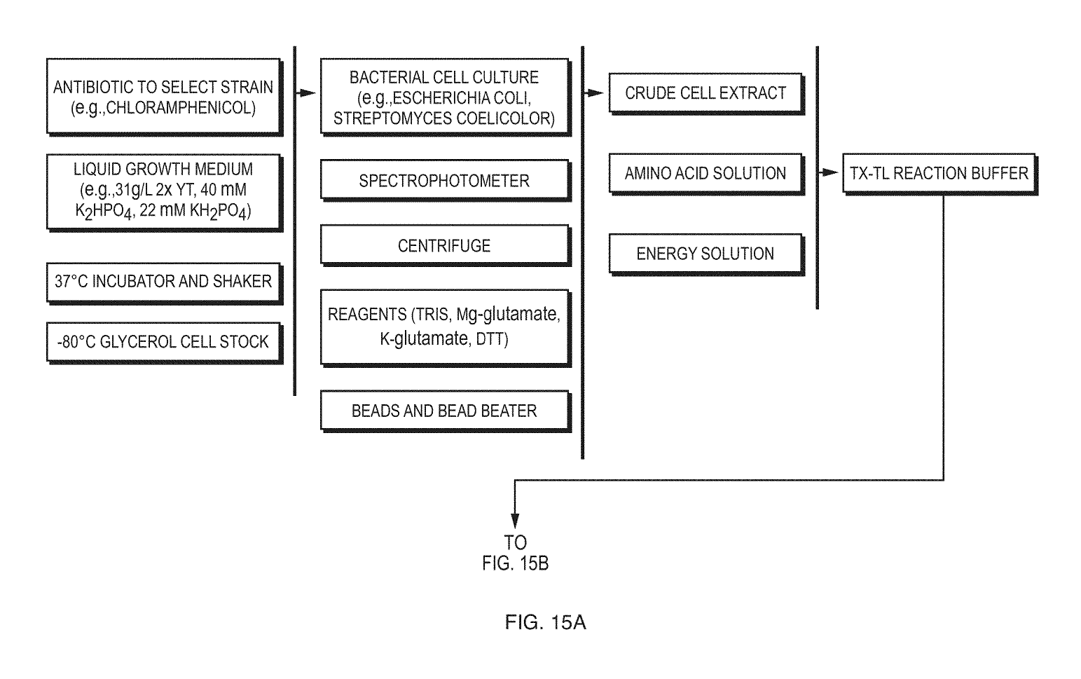

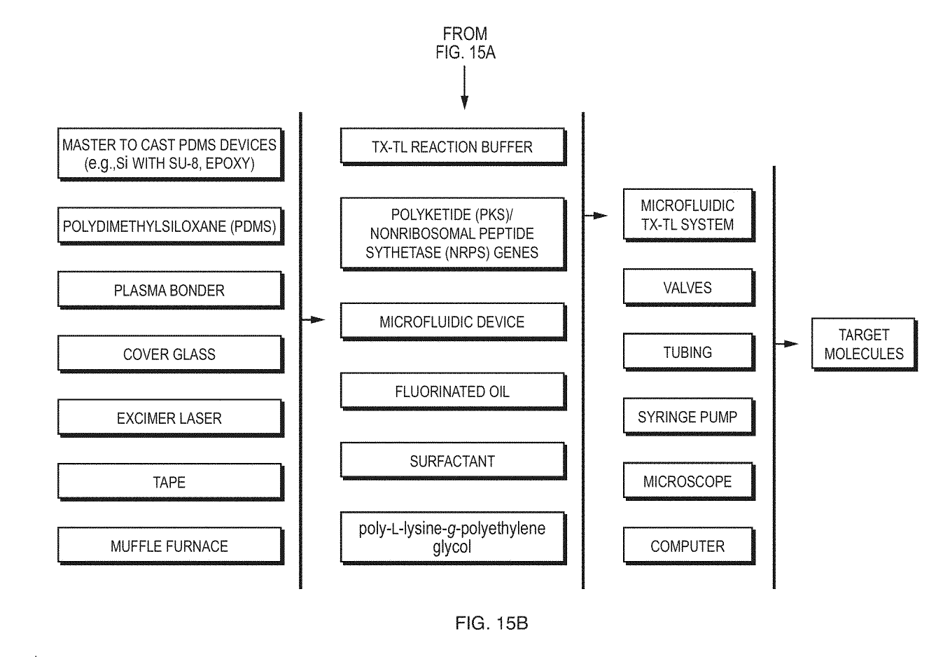

Alternatively, the gene expression library and modular hardware architecture are used separately. FIGS. 15A-B illustrate the independence of the two components of the system, wherein FIG. 15A depicts the options for an exemplary implementation of a cell-free TX-TL system according to one component of the system and FIG. 15B depicts options for an exemplary implementation of microfluidic cell-free biosynthesis according to the other component.

The gene expression library and modular hardware architecture each significantly increase capacity of cell-free biosynthesis. Each follows digital material design principles emphasizing component modularity and system adaptability at low cost. This results in a generalizable, robust platform technology. This generalizable platform can be used to optimize biosynthesis by heterologous expression in vivo or traditional cell-free expression.

Example Use Case: Molecular analysis and evaluation of polyketide activity against tumour cells. The lyngbyatoxin A (LTX) biosynthetic pathway is the smallest reported cyanobacterial non-ribosomal peptide (NRP) and LTX and structural analogues modulate protein kinase C and have been investigated in the treatment of cancers after standard molecular analysis of the generated molecules. This makes a good example case. Efficacy of different perturbations of the LTX pathway can be assayed by evaluating anticancer properties within the microfluidic platform. Thus, conditions that generate potent PK anticancer activity can be quickly identified. Numerous small molecule commercial fluorophores reveal cellular apoptosis or necrosis phenomena. These include cytochrome c translocation via calcein AM, calcein AM, membrane integrity via SYTOX® Green, and cellular metabolism via resazurin/resorufin. For long incubations, droplets can be stored in wells. Then the wells may be scanned for fluorescent indicators of PK anticancer activity with an automated microscope. Bright droplets will indicate active PKs. The gene ratios that generated these active PKs, are known as they can be programmed into the droplet compositions and sent to designated storage locations by the computer controlling the valving. With this information, in larger volumes the subset of potent cell-free TX-TL reactions can be repeated, to generate enough product for subsequent chemical and structural analyses. For short incubation protocols, the droplets can simply flow past an objective lens. As before, droplets can be generated in a user specified order, which enables correlating PK anticancer efficacy and PKS gene abundance.

Further, such a scheme not only further increases the number of droplet conditions per experiment, but also enables the ability to program new droplets in real time, to reproduce and modify potent PKs discovered earlier in the experiment. A continuous workflow, interpreted and guided by computational resources would allow adaptive iteration towards optimized PKs. For example, applied machine learning techniques to optimize cell-free systems with respect to various parameters that can be applied to this setting.

After gene combinations that generate potent PK are identified purifying them from the extract using a combination of analytical chemistry techniques can be used for characterization. Since the gene composition of each droplet has been programmed, the most potent reactions can be then reproduced in bulk mode using the same set of plasmids to generate enough yield for high-performance liquid chromatography, mass spectrometry and other molecular analysis techniques. Since independent measurements of the spectra of the PKSs using the same set of plasmids are made, it is possible to isolate the spectra of the unknown natural products created in the cell-free reactions.

Because the present invention follows digital material design principles emphasizing component modularity and system adaptability at low cost, it represents a generalizable, robust new technology of transformative potential, designed for quick adoption by other researchers and scale up necessary for adoption by industry. Each of the two component approaches significantly increases the capacities of conventional methods for cell-free biosynthesis. Used together, they create a transformative new capability for researchers and pharmaceutical producers, in the form of a generalizable technology platform. The invention therefore makes practical the production of known biosynthetic products (such as, but not limited to, cancer drugs) and dramatically accelerates the systematic search for new products (such as, but not limited to, medicines).

While several illustrative embodiments are disclosed, many other implementations of the invention will occur to one of ordinary skill in the art and are all within the scope of the invention. Furthermore, each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention, which is not to be limited except by the claims that follow.

What is claimed is:

1. An apparatus for cell-free synthesis of a biosynthetic product, comprising:
a cell-free transcription-translation system, comprising:
a cell culture apparatus configured for preparing a selected bacterial culture;
a cell extract generation apparatus configured for generating a cell extract from the bacterial cell culture;
a cell-free transcription-translation reaction buffer module configured for combining the cell extract with amino acid and energy solutions to create a cell-free transcription-translation reaction buffer; and
reaction buffer aliquots separation mechanism; and
a modular microfluidic system configured for generating, controlling, and manipulating droplets of the reaction buffer aliquots, the microfluidic system comprising one or more microfluidic modules, each microfluidic module comprising:
a plurality of ports configured to accept reaction buffer aliquots and dispense the droplets; and
at least one valve configured to control generation and manipulation of the droplets as they pass through the device.

2. The apparatus of claim 1, wherein the modular microfluidic system is controllable by a computer.

3. The apparatus of claim 1, wherein the microfluidic system further comprises at least one syringe pump for infusing the reaction buffer aliquots into at least one of the microfluidic modules.

4. The apparatus of claim 1, wherein the microfluidic system further comprises at least one identification device configured for identification of droplets containing target molecules.

5. The apparatus of claim 4, wherein the identification device comprises at least one microscope.

6. The apparatus of claim 1, wherein there are a plurality of microfluidic devices interconnected in parallel.

7. The apparatus of claim 1, wherein there are a plurality of microfluidic devices interconnected in series.

8. The apparatus of claim 1, wherein the reaction buffer aliquots are engineered to have different target molecules by adding a gene set to each buffer portion to create the reaction buffer aliquots, wherein at least one added gene set is different from at least one other added gene set, wherein the gene sets are part of a gene expression library comprising one or more gene sets, and wherein each gene set comprises the gene required for synthesis of a specific target molecule.

9. The apparatus of claim 1, wherein the microfluidic module is configured for performing at least one of the steps of:
generating droplets in specific ratios to program enzyme stoichiometries;
fusing droplets in specific ratios to program enzyme stoichiometries;
storing droplets within the microfluidic module;
incubating droplets within the microfluidic module;
analyzing the droplets based on performance assays performed within the microfluidic module; and
sorting the droplets based on performance assays performed within the microfluidic module.

10. An apparatus for cell-free biosynthesis, comprising:
a modular microfluidic device configured for generating, controlling, and manipulating droplets of reaction buffer aliquots in order to identify and separate target molecules, comprising:
a plurality of ports configured to accept reaction buffer aliquots and dispense the droplets;
at least one valve configured to control generation and manipulation of the droplets as they pass through the device; and
at least one identification device configured for identification of droplets containing target molecules.

11. The apparatus of claim 10, wherein the valves are controllable by a computer.

12. The apparatus of claim 10, wherein the identification device comprises a microscope.
